(19) United States
(12) Patent Application Publication
(10) Pub. No.: US 2022/0023139 A1
(43) Pub. Date: Jan. 27, 2022

(54) LUBRICATION DEVICE AND METHOD OF USE

(71) Applicants: Kyle J. Furey, Kansas City, MO (US); Christina Furey, Kansas City, MO (US)

(72) Inventors: Kyle J. Furey, Kansas City, MO (US); Christina Furey, Kansas City, MO (US)

(21) Appl. No.: 17/385,538

(22) Filed: Jul. 26, 2021

Related U.S. Application Data
(60) Provisional application No. 63/056,127, filed on Jul. 24, 2020.

(51) Int. Cl. A61H 19/00; A61F 13/40; B05C 3/09

(52) U.S. Cl. CPC A61H 19/00; A61M 35/006; B05C 3/09; A61H 2201/105; A61H 2205/087; A61H 2201/0153; A61H 2201/1697

(57) ABSTRACT

A lubrication device and method of use including a housing having an elongated, hollow tube or orifice configured for receiving a soft, porous material, the soft, porous material further including a central opening. The lubrication device further includes a removable end cap accommodating access to the housing inner orifice and porous material. In an exemplary embodiment, the end cap includes an access lid and an access lid opening. The soft, porous material of the present invention is configured for receiving and absorbing a lubricant, and the porous material central opening is configured for receiving a male penis, alternative human appendage, or a simulated male penis or human appendage for transfer of lubricant from the porous material onto the inserted actual or simulated body part. In an exemplary embodiment, the housing of the lubrication device has an outward appearance of an inconspicuous household item.

### CROSS-REFERENCE TO RELATED APPLICATION

This application claims priority in U.S. Provisional Patent Application No. 63/056,127, filed Jul. 24, 2020, which is incorporated herein by reference.

### BACKGROUND OF THE INVENTION

### 1. Field of the Invention

The invention relates generally to lubrication devices and methods of use thereof and more specifically, to a device configured for applying lubricant to a human reproductive organ, alternative human appendage, or simulated human organ or appendage prior to sexual intercourse to reduce friction and to facilitate more enjoyable intercourse.

### 2. Description of the Related Art

The female reproductive system naturally produces fluid, or lubrication, which helps promote reproductive health. When a woman is sexually aroused, her vagina produces extra lubrication. This extra lubrication helps to reduce friction during sexual intercourse and to minimize discomfort or irritation.

However, vaginal dryness is a very common issue for many women, leading to painful sexual intercourse, irritation, and/or soreness. Vaginal dryness can also lead to discomfort for a male sexual partner during sexual intercourse. Various medical conditions, medications, age, and/or other factors can hinder a woman's ability to naturally produce vaginal lubrication. Accordingly, many sexual partners utilize artificial sexual lubricants prior to sexual intercourse for a more enjoyable experience.

There are many types of sexual lubricants available for consumers. However, the application of such lubricants to reproductive organs can often be difficult, inefficient, and/or awkward. Additionally, use of a sexual lubricant often requires a user to dig through a drawer, cabinet, or other container to retrieve the lubricant prior to use because having a container of lubricant readily available on a nearby counter may be embarrassing for the user. What is needed is an inconspicuous device for easy and efficient application of a user's preferred lubricant to a human reproductive organ, human appendage, or simulated human organ or appendage.

Heretofore there has not been available a lubrication device and method of use with the advantages and features of the present invention.

### SUMMARY OF THE INVENTION

The present invention covers a lubrication device including a housing with an elongated, hollow tube or orifice configured for receiving a soft, porous material. In an aspect of the present invention, the device housing includes a removable end cap for enclosing and for allowing access to the internal porous material. With the end cap removed, the porous material is configured to receive a quantity of a desired sexual lubricant, and the porous material then absorbs the sexual lubricant. In an exemplary embodiment, the porous material further includes a central opening configured for receiving a male penis, alternative human appendage, or simulated human appendage. In alternative embodiments, the lubrication device includes two removable end caps, one on each end of the housing.

In use of an exemplary embodiment of the present invention, the end cap is removed, and a sexual lubricant is added into the device. The end cap is replaced, and the lubricant is absorbed into the soft, porous material. When ready, an end cap access lid on a proximal side of the end cap is opened, and a male penis, alternative human appendage, or simulated human appendage is inserted into the central opening in the saturated porous material. Alternatively, the entire end cap may be removed to allow access to the porous material. While inserted into the device, lubricant is transferred from the porous material onto the penis, alternative human appendage, or simulated human appendage. The actual or simulated human appendage is then removed from the device and is thus externally lubricated in anticipation of sexual intercourse. The end cap access lid may be closed or the end cap may be reattached to the lubrication device immediately or at a later time.

In some embodiments, the device includes a stopper piece having a central opening for insertion and removal of a male penis, alternative human appendage, or simulated human appendage therethrough but configured for retaining the soft, porous material within the device housing when the actual or simulated human appendage is removed. Such a stopper piece also helps to minimize leakage of lubricant. Other embodiments include a clasp or alternative attachment mechanism to help retain the porous material within the hollow tube or orifice.

In an exemplary embodiment, the soft, porous material is removable from the device for cleaning and/or replacement with a new piece of soft, porous material. Preferably, the outer housing can be hand washed and/or is dishwasher safe.

In a further aspect of the present invention, the external appearance of the housing may be a disguise so that a device embodying the present invention appears inconspicuous to visitors. Embodiments include a variety of disguised outer appearances.

### BRIEF DESCRIPTION OF THE DRAWINGS

The drawings constitute a part of this specification and include exemplary embodiments of the present invention illustrating various objects and features thereof.

FIG. 1 shows a top, front, perspective view of a lubrication device embodying the present invention.

FIG. 2 shows a side, elevational view of the lubrication device.

FIG. 3 shows a top, plan view of the lubrication device.

FIG. 4 shows a bottom, plan view of the lubrication device.

FIG. 5 shows a top, front, perspective view of the lubrication device with the top, access lid open.

FIG. 6 shows a side, elevational view of the lubrication device with the access lid open.

FIG. 7 shows a top, plan view of the lubrication device with the access lid open.

FIG. 8 shows a top, front, partially-exploded, perspective view of the lubrication device with the end cap removed from the housing and showing application of a lubricant to the inner, soft, porous material housed within the housing.

FIG. 9 shows a top, front, exploded, perspective view of the lubrication device.

FIG. 10 shows a side, exploded, elevational view of the lubrication device.

FIG. 11 is a side, elevational, cross-sectional view of the lubrication device.

FIG. 12 is a top, cross-sectional, plan view of the lubrication device.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right, and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Additionally, anatomical terms are given their usual meanings. For example, proximal means closer to the trunk of the body, and distal means further from the trunk of the body. Said terminology will include the words specifically mentioned, derivatives thereof and words of similar meaning.

### II. Preferred Embodiments

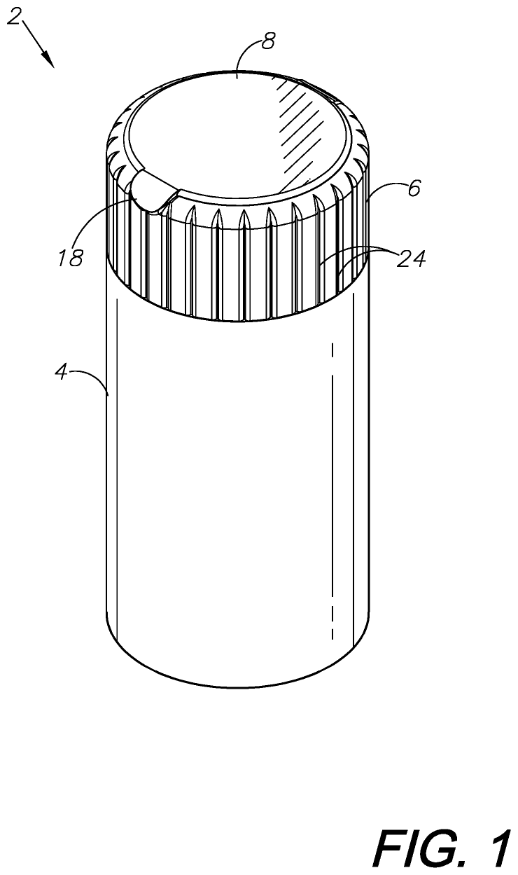

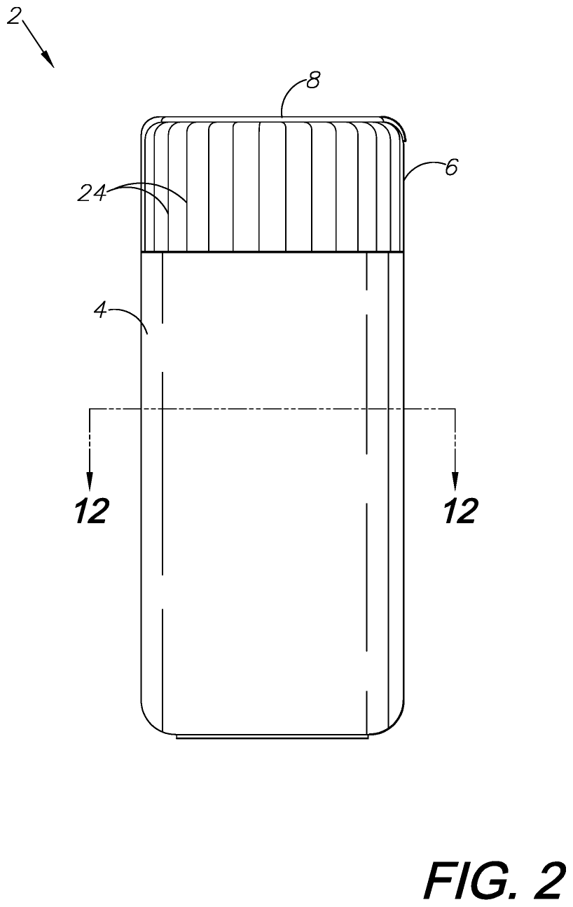

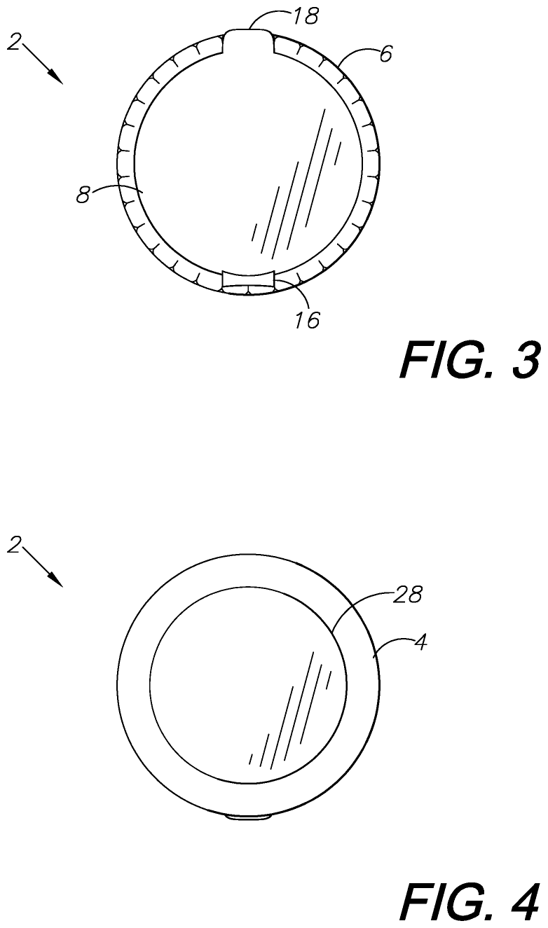

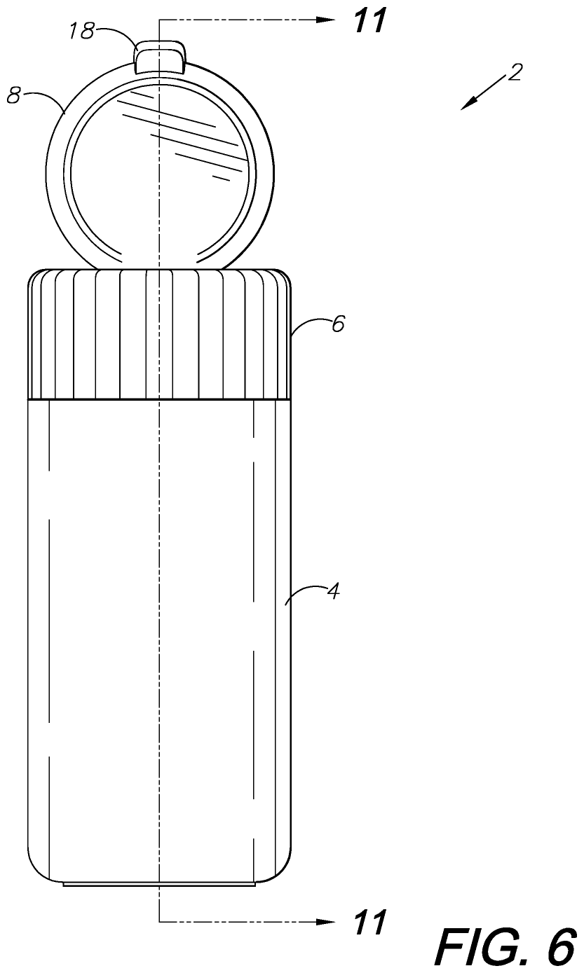

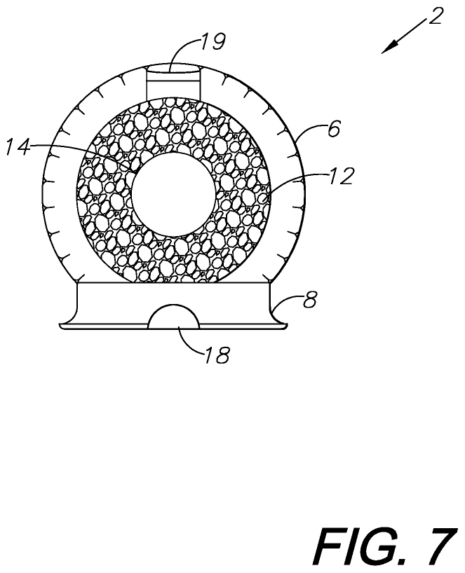

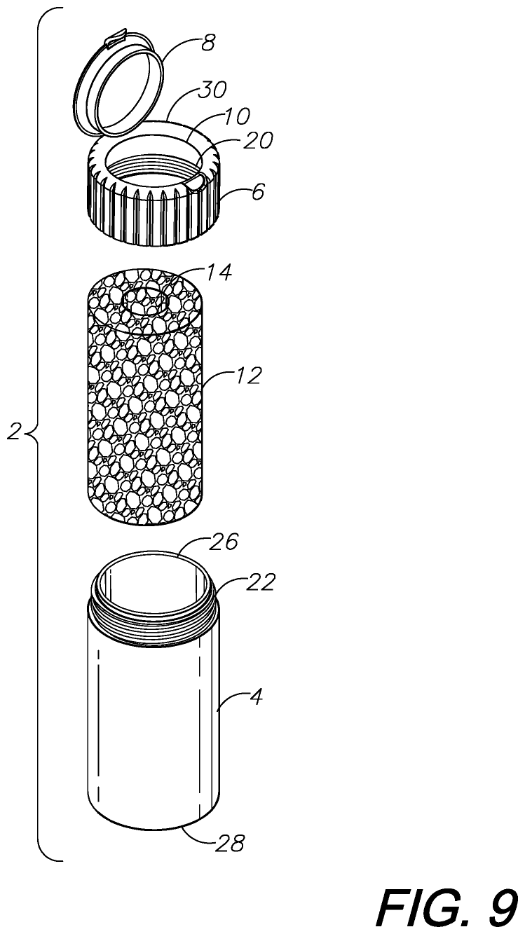

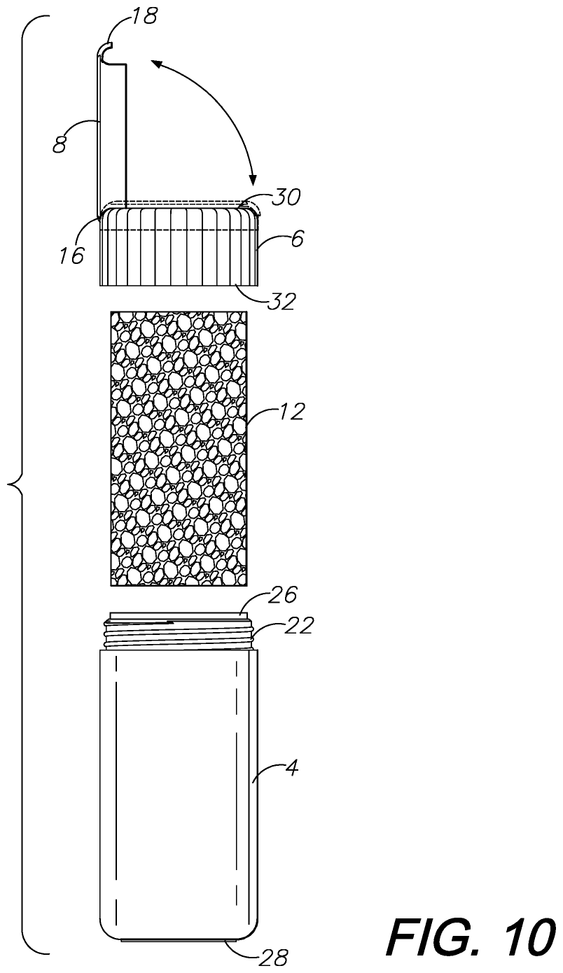

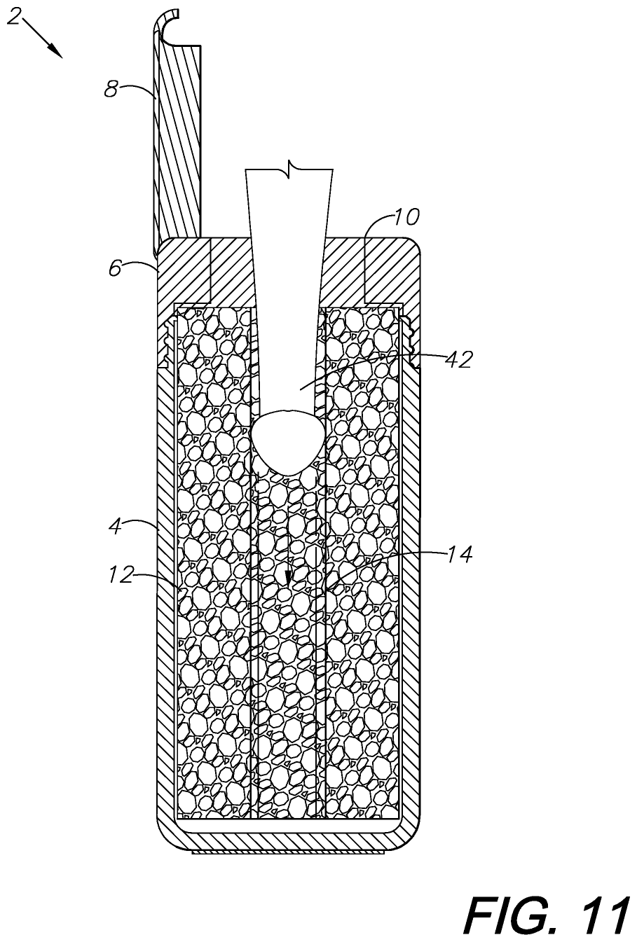

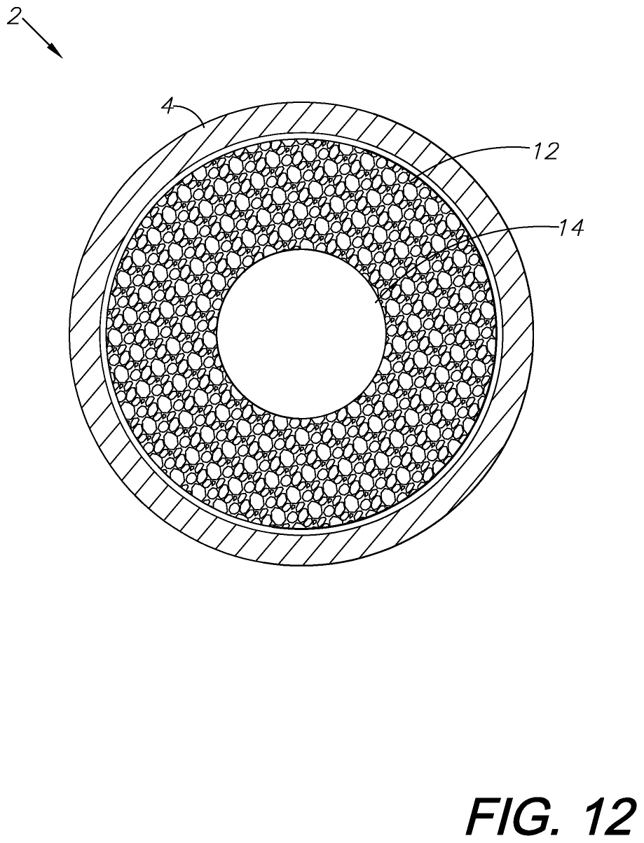

The present invention covers a lubrication device 2 and method of use. The device 2 includes an outer housing 4 having an elongated, hollow tube or orifice configured for receiving a soft, porous material 12, the soft, porous material 12 further including a central opening 14. The soft, porous material 12 of the present invention is configured for receiving and absorbing a lubricant 38, and the porous material central opening 14 is configured for receiving a male penis, alternative human appendage, or a simulated male penis or human appendage 42 for transfer of lubricant 38 from the porous material 12 onto the inserted actual or simulated body part 42. Referring to the drawings in more detail, FIGS. 1-12 show an exemplary embodiment of a lubrication device 2 of the present invention.

In an exemplary embodiment, the housing 4 is substantially cylindrical in shape with first and second ends: a proximal end and a distal end. However, in alternative embodiments, the housing 4 may form another shape having an inner orifice or cavity. In an exemplary embodiment, as shown in FIGS. 1-12, the housing 4 includes a removable end cap 6 at its proximal end 26 and an enclosed distal end 28. The proximal end removable end cap 6 encloses and allows access to the internal hollow tube or orifice and the housed porous material 12 of the present invention. The enclosed distal end 28, in this embodiment, helps prevent leakage of lubricant 38 from the housing 4. However, in alternative embodiments, the device 2 may have two removable end caps 6, one on each end of the housing 4. In such embodiments with two removable end caps, one end may be configured for filling the device 2 with personal sexual lubricant 38, and the opposite end may be configured for insertion of a reproductive organ, alternative appendage, or simulated body part 42.

In embodiments of the present invention, the mechanisms of removable attachment of the end cap(s) 6 to the main housing 4 include but are not limited to threaded or screw-on attachment, snap-in attachment, articulated attachment, compression rubber or silicone seal attachment, hook and loop fastener attachment, or any other type of attachment mechanism. In the embodiment shown in FIGS. 1-12, the mechanism of removable attachment is threaded attachment. The distal end 32 of the end cap 6 includes threads 20 configured for mating with threads 22 on the proximal end 26 of the housing 4. The exterior of the end cap 6 may further include grooves 24, as shown in FIGS. 1-3 and 5-10, to provide a better gripping surface for a user's hand. Alternatively, an additional material such as rubber or silicone may be added to the end cap 6 for a better grip surface. The end cap 6 of the lubrication device 2 may also optionally include an extended connection piece to the housing 4, similar to a water bottle having a lid extended connection piece, configured for allowing removal of the end cap 6 to provide access to the housing inner cavity but keeping a portion of the end cap 6 attached to the housing 4 via the connection piece.

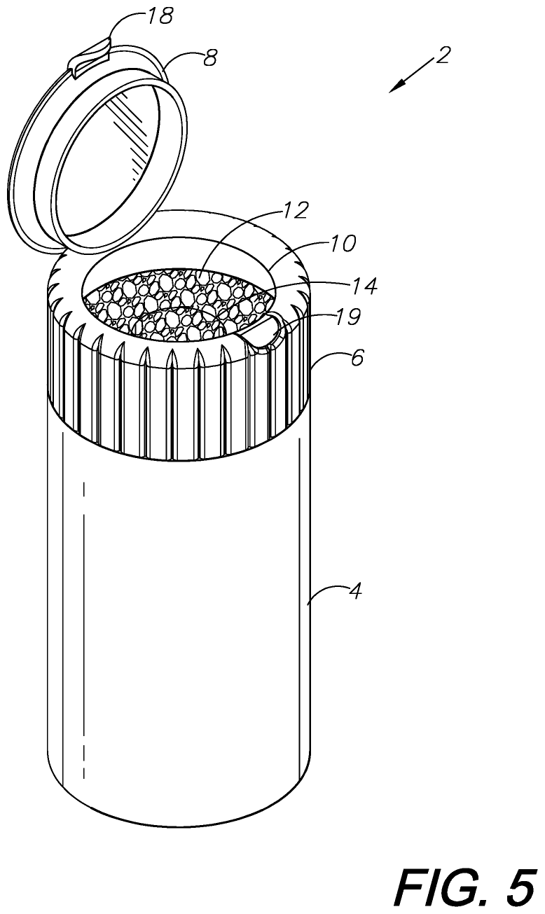

In a preferred embodiment, the removable end cap 6 further includes an access lid 8 at a proximal side 30 of the end cap 6 which opens and closes an access lid opening 10 separate from the attachment mechanism of the end cap 6 to the main housing 4. In an exemplary embodiment, the access lid opening 10 is smaller in diameter than the diameters of the end cap open distal side 32 and the main housing open proximal end 26. Preferably, at a minimum, the access lid opening 10 is approximately the same diameter as the diameter of the central opening 14 in the soft, porous material 12. The access lid opening 10 may also be larger in diameter than the diameter of the porous material central opening 14 but smaller in diameter than the main housing 4 open proximal end 26, as shown in FIGS. 5 and 7-9. Such a configuration accommodates comfortable and easy insertion of a male penis, other human appendage, or simulated body part 42 through the access lid opening 10 and into the central opening 14 in the porous material 12 for transfer of personal lubricant 38 onto the actual or simulated body part 42. The configuration further results in the inner porous material 12 being retained within the housing 4 cavity by the remainder of the proximal side 30 of the end cap 6 surrounding the access lid opening 10 when the body part or simulated body part 42 is removed from the housing 4 cavity through the access lid opening 10.

The access lid 8 of the present invention includes an open and close attachment mechanism for selective opening and closure of the access lid opening 10 via connection to the rest of the end cap 6. In an exemplary embodiment, the access lid 8 includes a releasable clasp 18 for connection to a clasp receiver 19 positioned on the end cap 6 when closed. Alternatively, the clasp 18 may be configured for connection to the rest of the end cap 6 itself without a designated clasp receiver 19. Embodiments of an access lid clasp 18 include a compression clasp, a spring-loaded clasp, or any other type of releasable clasp. Alternatively, the access lid 8 may attach to the rest of the end cap 6 via snap-in attachment, articulated attachment, compression rubber or silicone seal attachment, hook and loop fastener attachment, or any other type of attachment mechanism. In preferred embodiments of the lubrication device 2, the access lid 8 includes a hinged or alternative connection piece 16 configured for keeping the access lid 8 attached to the end cap 6 when the access lid opening 10 is opened to allow access to the porous material central opening 14. However, in alternative embodiments the access lid 8 is fully detachable from the rest of the end cap 6. In further embodiments of the present invention, the lubrication device 2 does not include an access lid 8 and access lid opening 10 and instead requires removal of the end cap 6 for application of lubricant 38, insertion of an actual or simulated body part 42 into the porous material central opening 14, and removal of the porous material 12 as needed.

In a preferred embodiment, the outer housing 4 is made up of a rigid material. However, alternatively, the outer housing 4 may have some elasticity. The housing 4 may be made up of, but is not limited to, plastic, rubber, composite materials, synthetic textiles, metal, plaster, or wood.

The soft, porous material 12 is configured for receiving and absorbing a quantity of a sexual lubricant 38 of the user's choice and for transferring lubricant 38 onto a penis, alternative appendage, or simulated body part 42 which makes contact with it. Any type of liquid lubricant 38 can be used with embodiments of the present invention. The soft, porous material 12 must be absorbent, open-cell, compressible, and firm enough to be able to receive and transfer lubricant 38 to an inserted actual or simulated body part 42 making contact, but the material 12 must also be soft enough to not cause injury or discomfort to the inserted actual or simulated body part 42. Additionally, the porous material 12 must be soft enough to transfer lubricant 38 to a male reproductive organ without causing sexual stimulation of the inserted male reproductive organ. The purpose of the present device 2 is to provide external lubrication to a male penis, alternative human appendage, or simulated penis or appendage 42 prior to sexual intercourse while avoiding sexual stimulation during the lubrication process prior to said sexual intercourse. Embodiments of the soft, porous material 12 include, but are not limited to, natural sponge material, artificial sponge material, and soft foam material.

In an exemplary embodiment, the soft, porous material 12 includes a central opening 14 open at the proximal end of the porous material 12 and extending through the majority, if not all, of the length of the porous material 12 toward the distal end of the porous material 12. The porous material central opening 14 is configured for receiving a male penis, alternative human appendage, or simulated body part 42. The central opening 14 in the porous material 12 does not include any sort of ribbings to help prevent male sexual stimulation. In embodiments of the present invention, porous material 12 of the lubrication device 2 may be available with different sizes of central opening 14, as desired, based on the size of the actual or simulated body part 42 to be inserted and lubricated.

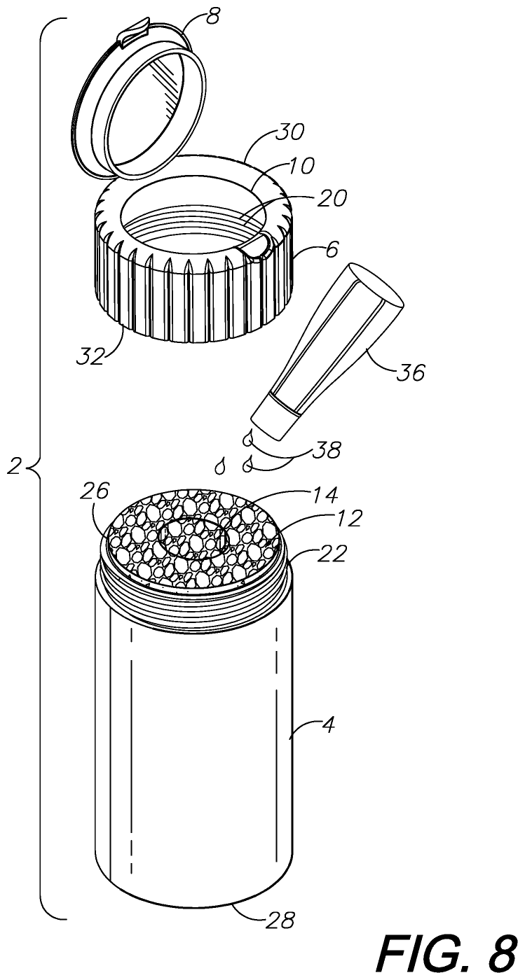

In use of an exemplary embodiment of the lubrication device 2, the end cap 6 of the lubrication device 2 is removed from the main housing 4, and a quantity of sexual lubricant 38 of the user's choice is added onto the soft, porous material 12 within the housing 4 inner cavity from a squeeze bottle 36 or similar alternative container sold retail, as shown in FIG. 8. The end cap 6 is reattached to the housing proximal end 26, and the lubricant 38 is absorbed by the soft, porous material 12. In anticipation of sexual intercourse, a user opens the access lid 8, and a male penis, alternative human appendage, or simulated body part 42 is inserted through the access lid opening 10 and into the central opening 14 in the lubricant-saturated, porous material 12. With the actual or simulated body part 42 inserted into the device 2, lubricant 38 is transferred from the porous material 12 onto the actual or simulated body part 42. The actual or simulated body part 42 is then removed from the device 2, resulting in sexual lubricant 38 being externally applied to the actual or simulated body part 42 in preparation for sexual intercourse. In a preferred embodiment, the rest of the proximal side 30 of the end cap 6 surrounding the access lid opening 10 retains the soft, porous material 12 within the housing 4 cavity as the actual or simulated body part 42 is removed. The access lid 8 can either be closed immediately or at a later time, as desired. If the access lid 8 is to be closed later, the device 2 is placed with the open lid facing upward to prevent leakage of lubricant 38.

The present device 2 is configured for application of sexual lubricant 38 to a human reproductive organ or appendage 42 in anticipation of sexual intercourse and not for masturbation or sexual stimulation from insertion of a human reproductive organ 42 into the device 2 and/or removal of the human reproductive organ 42 from the device 2. The soft, porous material 12 is preferably overly soft and absorbent to facilitate efficient transfer of lubricant 38 and not to provide sexual stimulation from insertion into the porous material 12 nor to simulate insertion into a female or male orifice.

In some embodiments of the present invention, the lubrication device 2 may further include a stopper piece, to help retain the soft, porous material 12 within the device 2 when a penis, alternative human appendage, or simulated body part 42 is removed and to help minimize leakage of lubricant 38. Such a stopper piece is particularly useful for embodiments without a separate access lid opening 10 on the end cap 6. In such embodiments, the stopper piece includes a stopper central opening to accommodate insertion and removal of a male penis, alternative human appendage, or simulated body part 42 therethrough. Preferably, the stopper piece central opening is soft and expandable for comfortable insertion and removal of an actual or simulated body part 42. Additional embodiments include an attachment mechanism to help retain the porous material 12 within the housing 4 hollow tube or orifice when a penis, alternative human appendage, or simulated body part 42 is removed from the device 2. Such attachment mechanisms include a clasp, hook and loop fasteners, or any other type of attachment mechanism.

In an exemplary embodiment of the lubrication device 2 of the present invention, the soft, porous material 12 is removable from the hollow tube or orifice. Preferably, the porous material 12 may be removed to be cleaned and reinserted into the housing 4 hollow tube or orifice. Alternatively, the soft, porous material 12 may be disposable and configured to be replaced with a new piece of soft, porous material 12 after one or more uses, as desired. In further embodiments, pieces of the soft, porous material 12 may be individually packaged and pre-lubricated. In an exemplary embodiment, the outer housing 4 is washable by hand washing and/or by dishwasher.

In an exemplary embodiment, the external appearance of the housing 4 provides a disguise such that the device appears inconspicuous to any visitors or passersby. In a preferred embodiment, as shown in FIGS. 1-12, the outer appearance of the lubrication device 2 looks like a water bottle or beverage container. In further embodiments, the external appearance of the housing 4 may resemble a candle, a vase, a decorative container, a diffuser, a statue, a figurine, a flashlight, or any other inconspicuous household item.

In a further embodiment of the present invention, the center of the porous material 12 includes a removable, rigid rod or member with attached additional porous material 12. This rod or member with attached additional porous material 12 could be attached to an end cap 6 or to a separate handle. Such an embodiment with a rod or member with attached additional porous material 12 is configured to accommodate insertion into a female vagina or other human orifice to apply sexual lubricant thereto. The removed rod or member would provide an opening 14 for insertion of a male penis, alternative human appendage, or simulated body part 42 into the rest of the porous material 12.

In additional embodiments, the present invention may be adapted for applying alternative types of lubricant to items needing lubrication, such as but not limited to metal tools. In such an embodiment for lubrication of metal tools, the device 2 is configured for insertion of a metal tool into a central opening 14 in porous material 12 saturated with a metal lubricant in the same manner as insertion of an actual or simulated body part 42 into a central opening 14 in porous material 12 saturated with sexual lubricant 38 as described above.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. A lubrication device comprising:
a housing having first and second ends and an inner cavity open at said housing first end;
an end cap configured for removable attachment to and for enclosing said housing first end;
a porous material having first and second ends and configured for being housed within said housing inner cavity;
said porous material comprising a central opening open at said porous material first end and extending a length through said porous material toward said porous material second end;
said porous material configured for receiving a fluid;
said porous material central opening configured for receiving an object to be inserted; and
said porous material configured for transferring said fluid onto said object.

2. The lubrication device according to claim 1, wherein:
said end cap further comprises an access lid and an access lid opening;
said access lid configured for accommodating selective opening and closure of said access lid opening; and
said access lid opening configured for allowing access into said porous material central opening.

3. The lubrication device according to claim 2, wherein:
said access lid opening is smaller than said housing open first end.

4. The lubrication device according to claim 2, wherein:
said access lid further comprises a clasp;
said end cap further comprises a clasp receiver; and
said access lid clasp is configured for releasable attachment to said end cap clasp receiver.

5. The lubrication device according to claim 1, wherein:
said porous material comprises an open-cell porous material.

6. The lubrication device according to claim 1, wherein:
said porous material central opening is approximately cylindrical in shape.

7. The lubrication device according to claim 1, wherein:
said fluid comprises a sexual lubricant; and
said object comprises an actual or simulated body part.

8. The lubrication device according to claim 7, wherein:
said porous material comprises a material soft enough to avoid sexual stimulation of said actual or simulated body part.

9. The lubrication device according to claim 1, wherein:
said housing further comprises an outer appearance of an inconspicuous household item.

10. The lubrication device according to claim 1, further comprising:
a stopper piece having a stopper central opening and configured for placement between said porous material first end and said housing first end; and
wherein said stopper piece is configured for retaining said porous material within said housing inner cavity when said object is removed from said device.

11. The lubrication device according to claim 1, wherein:
said housing is open at said housing second end; and
said lubrication device further comprises a secondary end cap configured for removable attachment to and for enclosing said housing second end.

12. The lubrication device according to claim 1, wherein:
said fluid comprises a metal lubricant; and
said object comprises a metal tool.

13. A lubrication device comprising:
a housing having first and second ends and an inner cavity open at said housing first end;
an end cap configured for removable attachment to and for enclosing said housing first end;
said end cap further comprising an access lid and an access lid opening;
said access lid configured for accommodating selective opening and closure of said access lid opening;
wherein said access lid opening is smaller than said housing open first end;
a porous material having first and second ends and configured for being housed within said housing inner cavity;
said porous material comprising a central opening open at said porous material first end and extending a length through said porous material toward said porous material second end;
said porous material configured for receiving a sexual lubricant;
said porous material central opening configured for receiving an actual or simulated body part to be inserted;
said porous material configured for transferring said sexual lubricant onto said actual or simulated body part; and
said housing further comprising an outer appearance of an inconspicuous household item.

14. A lubrication method using a lubrication device including a housing having first and second ends and an inner cavity open at the housing first end; an end cap configured for removable attachment to and for enclosing said housing first end; and a porous material having first and second ends and a central opening open at the porous material first end and extending a length through the porous material toward said porous material second end, the method comprising the steps of:
removing said end cap from said housing first end;
placing said porous material within said housing inner cavity;
applying a fluid to said porous material;
said porous material absorbing said fluid;
inserting an object into said porous material central opening;
said porous material transferring said fluid onto said object; and
removing said object from said lubrication device.

15. The lubrication method according to claim 14, wherein the lubrication device end cap further comprises an access lid and an access lid opening, the method further comprising the steps of:
reattaching said end cap to said housing first end;
opening said access lid; and
wherein said inserting an object into said porous material central opening comprises inserting said object through said access lid opening and into said porous material central opening.

16. The lubrication method according to claim 15, further comprising the step of:
said end cap retaining said porous material within said housing inner cavity when said object is removed from said lubrication device.

17. The lubrication method according to claim 15, further comprising the step of:
closing said access lid.

18. The lubrication method according to claim 14, wherein:
said fluid comprises a sexual lubricant; and
said object comprises an actual or simulated body part.

19. The lubrication method according to claim 14, further comprising the steps of:
removing said porous material from said housing inner cavity; and
washing said lubrication device.

20. The lubrication method according to claim 14, further comprising the step of:
reattaching said end cap to said housing first end.
